(19) United States
(12) Patent Application Publication
(10) Pub. No.: US 2018/0237359 A1
(43) Pub. Date: Aug. 23, 2018

(54) HYBRID CHEM-BIO METHOD TO PRODUCE DIENE MOLECULES

(71) Applicant: Technology Holding, LLC, Salt Lake City, UT (US)

(72) Inventors: Mukund R Karanjikar, SANDY, UT (US); Mahesh V Bule, Salt Lake City, UT (US); Robert A Price, Cottonwood Heights, UT (US)

(73) Assignee: Technology Holding, LLC, Salt Lake City, UT (US)

(21) Appl. No.: 15/753,331

(22) PCT Filed: Aug. 18, 2016
(86) PCT No.: PCT/US16/47576

§ 371 Date: Feb. 18, 2018

Related U.S. Application Data
(60) Provisional application No. 62/207,349, filed on Aug. 19, 2015.

(51) Int. Cl. C07C 1/24; C07C 11/08; C07C 11/10; C12P 7/18; C12P 7/06; C12P 7/16

(52) U.S. Cl. CPC C07C 1/24; C07C 11/08; C07C 11/10; C12P 7/18; C07C 2521/04; C12P 7/16; C07C 2529/40; C07C 2531/08; C07C 2523/10; C12P 7/06

(57) ABSTRACT

A method 100 to produce one or more diene molecules 135 including steps of preparing a biomass hydrolysate 137 from biomass, producing an engineered organism 120 that can feed on the biomass hydrolysate and express an alcohol product useful to make the diene molecule, fermenting 115 the broth with the engineered organism, separating 125 the alcohol product from fermentation broth, and catalyzing 130 the alcohol to create the diene molecule.

### PRIORITY CLAIM

This application is the National Phase entry of PCT/US16/047576, filed Aug. 18, 2016, and claims the benefit under 35 U.S.C. 119(e) of the filing date of U.S. Provisional Patent Application Ser. No. 62/207,349, filed on Aug. 19, 2015, and titled “Hybrid Chem-Bio Process for Production of Isoprene”, the entire contents of which are incorporated by this reference as though set forth herein in their entirety.

### TECHNICAL FIELD

This invention relates generally to processes for creating or producing diene molecules, nonexclusively including isoprene and butadiene. A preferred embodiment provides a hybrid chemical-biological (chem-bio) process to that effect.

### BACKGROUND

Dienes including isoprene and butadiene are predominantly produced from light naphtha cracking. In that process, narrow boiling range (71-104° C.) light naphtha is fed to an ethylene cracker with high-pressure hydrogen at high temperature and pressure (e.g., 500° C., 50 atm). Isoprene and butadiene are produced as minor compounds during ethylene production. Butadiene and isoprene may be separated from the process stream with elaborate separation schemes, such as multiple distillation steps. As an example, for a cracker of 1 MMTPA ethylene, only 20,000 tonnes of isoprene is co-produced. This corresponds to a paltry 2% yield.

Pyrolytic gasoline production applies steam cracking of heavy naphtha or light hydrocarbons, such as propane or butane, to produce ethylene. The yield is a liquid by-product rich in aromatic content called pyrolysis gasoline. This process also co-produces Isoprene at 1% or lower yield. Isoprene may be separated from the mixture via solvent extraction and distillation.

Certain other processes have been at an R&D level for many years, but are not yet feasible on a commercial scale. These R&D processes include pentane conversion, propylene dimerization & cracking, butene hydroformylation, acetone-acetylene reaction, isopentane double dehydrogenation, and isobutene-formaldehyde reaction. Obstacles in the way of commercialization include very low selectivity and low yields (isopentane dehydrogenation), high severity of operation, expensive feedstock (formaldehyde, acetone), hazardous operations (acetylene), and high capital costs (butene hydroformylation).

Multiple methods for producing a complete biologic pathway to isoprene have been known since the mid 1990's. Initially inventors isolated and cultured microorganisms that naturally produced isoprene (as disclosed in U.S. Pat. No. 5,849,970A), followed shortly thereafter by the transfer of a plant based isoprene synthase gene into bacteria (as disclosed in WO1998002550A2). More recently, companies such as Goodyear Tire and Rubber Co., DuPont, and Danisco US Inc. have developed variants of the isoprene synthase gene for more efficient production in microbial systems (see US20140234926A1, WO2010031077A1, and US20140128645A1). In all these cases, the base metabolic pathway harnessed was the mevalonate pathway. Despite 20 years of work on isoprene production through the mevalonate pathway, it is believed that no one has investigated a hybrid biologic-chemical approach to producing isoprene.

U.S. Pat. No. 7,985,567 discloses methods for bio-synthesis of branched 5-carbon alcohols, the entire disclosure of which is hereby incorporated by reference as though set forth herein in its entirety.

### DISCLOSURE OF THE INVENTION

The present invention provides a process for production of diene molecules. In particular, one exemplary such process includes the steps of deconstructing a carbon-bearing biomass to form monomeric sugars called biomass hydrolysate, fermenting a broth containing biomass hydrolysate with an engineered organism that expresses a desired precursor alcohol operable as a building block for one or more target diene molecules, separating the alcohol from the fermented broth, and catalytically converting the alcohol into one or more diene molecules.

### BRIEF DESCRIPTION OF THE DRAWINGS

In the drawings, which illustrate what are currently regarded as the best modes for carrying out the invention and in which like reference numerals refer to like parts in different views or embodiments:

FIG. 1 is a schematic illustrating a process according to certain principles of the invention;

FIG. 1A is a schematic illustrating additional details of an exemplary process according to certain principles of the invention;

FIG. 2 illustrates the mevalonate pathway for methylbutenol production from isoprenyl diphosphate (IPP);

FIG. 3 is a bar chart showing acetate production in the engineered strain 3A (3A Strain) and reduced production in ackA knockout strain 3A (3A ackA KO);

FIG. 4 is a schematic illustrating a metabolic diagram of butanediol (BDO) production;

FIG. 5 is a schematic illustrating overexpression of the BDO producing operon

FIG. 6 is an X-Y plot showing a high pressure liquid chromatography (HPLC) chromatogram of biomass hydrolysate (Peaks at 10.7 and 11.47 min—glucose & xylose resp. Sugar concentrations: 262 g/L of glucose and 108 g/L xylose, yielding a ratio of 2.43:1);

FIG. 7 is a bar chart showing methylbutenol titer of minimal media trials supplemented with 10 g/L of glucose compared to the rich media standards EZ rich and LB media in shake flask conditions;

FIG. 8 is an X-Y plot illustrating fermentation of strain KG1R10 in MOPS minimal media, in which total methylbutenol yield was 6.12 g/L at an efficiency of 58% of theoretical maximum yield;

FIG. 9 is an X-Y plot illustrating fermentation of strain KG1R10 in MOPS minimal media with further optimized protocol and secondary capture mechanisms to minimize loss due to entrainment and evaporation, in which total methylbutenol yield was 10.02 g/L at an efficiency of 65% of theoretical maximum yield;

FIG. 10 is an X-Y plot illustrating formation of BDO and lactate as parallel processes;

FIG. 11 is an X-Y plot illustrating the elimination of lactate through directed natural selection;

FIG. 12 is a schematic illustrating a two-stage extraction scheme for recovery of methylbutenol from water;

FIG. 13 is a bar chart illustrating methylbutenol conversion to isoprene as a function of temperature;

FIG. 14 is an X-Y plot illustrating methylbutenol conversion to isoprene as a function of flow rate;

FIG. 15 is a schematic illustrating a workable apparatus for BDO conversion to butadiene;

FIG. 16 is an X-Y plot showing BDO conversion to Butadiene and 2-butene (Scandium oxide, alumina composite bed) at a Hydrogen:BDO ratio of 4, T=250° C.; and

FIG. 17 is a bar chart illustrating BDO demonstrated molar conversion to 1,3-butediene using different catalysts: SC: scandium, SC+ZSM5: scandium and ZSM5 Zeolite, C/D-SC_AL: concentrated/dilute scandium immobilized on alumina, SC+AL_Sbed: Scandium and Alumina oxide separate bed, SC+AL_Mix: Scandium and Alumina Oxide mixture.

### MODES FOR CARRYING OUT THE INVENTION

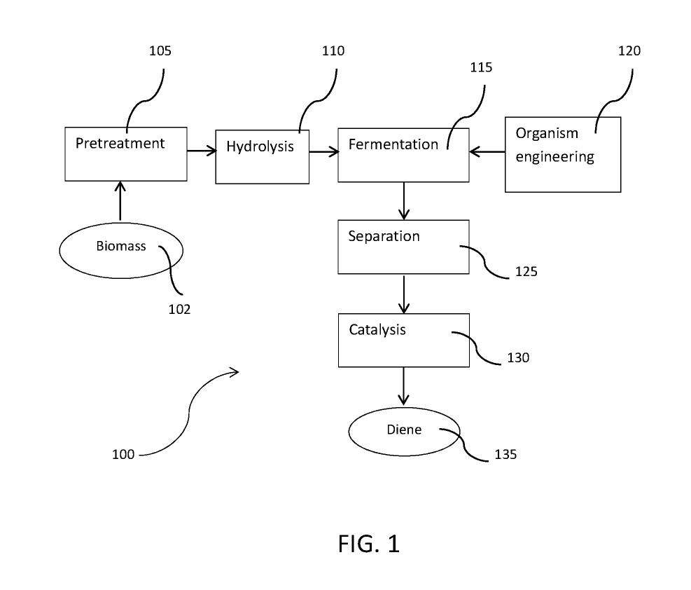

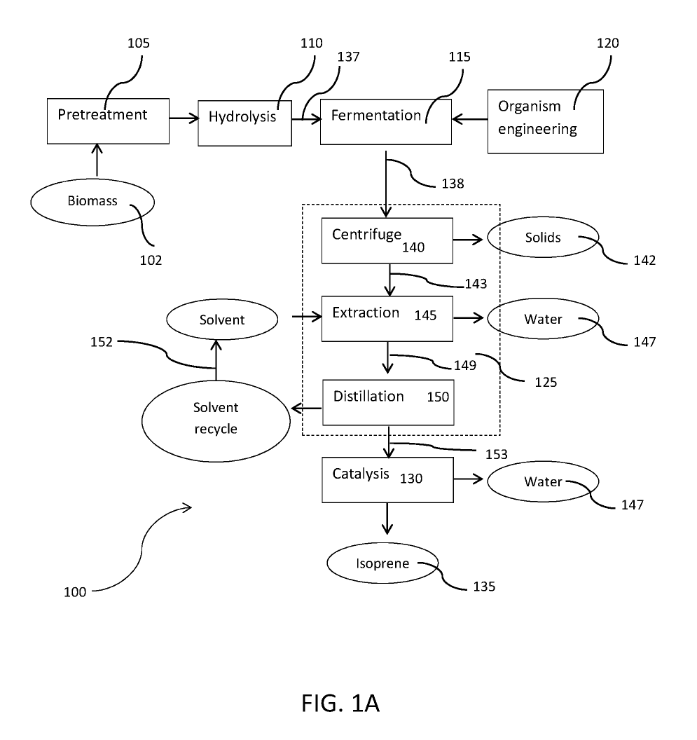

A method according to certain principles of the invention is shown in FIG. 1, and is generally indicated at 100. The method 100 includes conversion of biomass 102 to dienes via a hybrid fermentation and catalysis approach. A workable biomass 102 provides a suitable carbon source feedstock, which is typically pretreated, as indicated at block 105, and hydrolyzed, as indicated at block 110, to release monomeric five-carbon (C5) and six-carbon (C6) sugars. This carbon carrying feedstock undergoes a fermentation step, as indicated at block 115, to produce an alcohol. An exemplary product alcohol may have a minimum of two functional groups or have two hydroxyl groups. Desirably, a product alcohol will have at least two different reactive sites for further conversion. In one particular embodiment 100, the produced alcohol is 2,3-butanediol, whereas in another embodiment 100 the produced alcohol is methylbutenol. Additional embodiments 100 may be constructed to produce additional and alternative diene molecules. The production of the target alcohol product is dependent upon type and extent of engineering of the chosen microorganism, indicated at block 120. The product alcohol (e.g., 2,3-butanediol or methylbutenol), is then separated via a single step or a combination of multiple steps as indicated at block 125. The separated alcohol is then reacted over a catalyst bed to convert it to a corresponding diene, as indicated at block 130. For example, catalytic conversion of 2,3-butanediol produces butadiene in a single step whereas catalytic conversion of methylbutenol produces isoprene.

FIG. 1A illustrates an exemplary process 100 adapted to produce isoprene. A biomass feedstock 102 is obtained and put through a pretreatment step 105 to initiate breakdown of the biomass. Pretreatment step 105 may include treatment with acids, alkali, water, ammonia, organic solvents, carbon dioxide, lime or any combinations thereof at various temperature and pressure conditions. Subsequent to the pretreatment step 105, a hydrolysis procedure 110 is performed to form monomeric sugars which are then added as a biomass hydrolysate 137. A workable hydrolysis step 110 may include, or be effected by way of addition of an enzyme or a set of enzymes to produce monomeric sugars for a liquid biomass hydrolysate. That biomass hydrolysate undergoes a fermentation step 115 in which a product is an alcohol. Typically, an engineered organism is produced in an organism engineering step 120, where the organism is typically engineered to improve expression of a desired alcohol product. The obtained organism is then incorporated into the fermentation step 115. An exemplary engineered organism includes strain 3A E. coli with mevalonate pathway and added hydrolase NudB to convert Isoprenyl diphosphate (IPP) to methylbutenol. Fermented broth 138 is run through a separation step 125. A workable separation step 125 includes a centrifuge step 140, in which the bacteria and other solid materials 142 are centrifugally separated from the fluid portion 143 of the fermented broth. The fluid portion 143 may simply be decanted in preparation for the solvent extraction step 145. Solvent extraction step 145 includes adding an organic solvent, such as benzene, to the fluid portion 143 to extract to the alcohol product, and leave behind excess water 147. Produced water 147 may be incorporated into the hydrolysis step 110, if desired. The solvent and alcohol blend 149 may be processed in a distillation step, or by temperature-based selective evaporation and collection by condensation. Solvent recovery can approach 100%, and the solvent may be recycled as indicated at arrow 152. The captured and isolated alcohol product 153 is then processed in the catalysis step 130 to obtain the diene 135, in this case isoprene. Water 147 formed as a side product of the catalysis step may also be incorporated into the hydrolysis step 110, if desired.

Operable feedstock material, or biomass 102, nonexclusively includes hexose, pentose, cellulose, hemicellulose, cellobiose, glycerol, lactose, sucrose, woody biomass, corn stover, wheat straw, forestry residue, farm waste, and purpose-grown energy crops. Exemplary purpose-grown energy crops include sorghum, miscanthus, and switchgrass. Operable woody biomass further includes all trees, plants and shrubs. Another operable carbon source includes municipal solid waste. Other feedstock candidates include glycerol, mixture of hydrogen and carbon monoxide, methane, methanol and/or hydrocarbons.

The fermentation step 115 may be aerobic or anaerobic performed in a stirred or non-stirred vessel. The fermentation step 115 may further include solid-state fermentation.

Organisms used for the fermentation step 115 nonexclusively include one or more organism that may be selected from prokaryotic and eukaryotic organisms. Useful organisms for the fermentation step 115 may include but are not limited to bacteria, yeast, fungi, archaea, cyanobacteria, insect, plant, and mammalian cells. An operable organism for the fermentation step 115 may include gram-positive bacterial cells, gram-negative bacterial cells, filamentous fungal cells, algae cells, and yeast cells. Certain operable organisms for the fermentation step 115 may be selected from Escherichia sp. (E. coli), Panteoa sp. (P. citrea), Bacillus sp. (B. subtilis), Yarrowia sp. (Y. lipolytica), Saccharomyces sp. (S. cerevisiae), Pichia sp. (P. pastoris), Trichoderma sp. (T. reesei), Aspergillus sp. (A. oryzae or A. niger), Klebsiella sp. (K. oxytoca or K. pneumoniae), Streptomyces sp. (S. lividans or S. californicus), Clostridium sp. (C. ljungdahlii), Enterobacter sp. (E. aerogenes), Aerobacillus sp. (A. polymyxa), Lactococcus sp. (L. lactis), Paenibacillus sp. (P. polymyxa), Serrati sp. (S. marcescens), Candida sp. (C. rugosa), Geobacillus sp. (G. thermoglucosidasius), Serratia sp. (S. plymuthica), Pyrococcus sp. (P. furiosus), Corynebacterium sp. (C. glutamicum), and Pseudomonas sp. (P. aeruginosa). It is generally preferred that the organism(s) used in the fermentation step is/are engineered to increase production of a desired target alcohol over its/their wild or pre-engineered condition.

A workable separation step 125, to separate alcohol product from fermentation broth, may nonexclusively include one or more of the following procedures: distillation, filtration, solvent extraction, membrane separation, pervaporation, absorption, adsorption, vacuum distillation and/or use of adducts. In case of solvent extraction being one of the separation procedures, exemplary solvents that can be used for extraction nonexclusively include methyl iso-butyl ketone, methyl ethyl ketone, acetone, ethanol, propanol, hexane, butyl acetate, ethyl acetate, benzene, toluene, xylene, N-Methyl-2-pyrrolidone, glycerol, glycol, cyclohexane, chloroform, dichloromethane, ethyl acetate, dimethyl formamide, acetonitrile, dimethyl sulfoxide and butanol.

Conversion of product alcohol to the corresponding diene can be performed by catalysis in a continuous stirred tank or packed bed reactor. The catalysis step 130 can be performed in the temperature range of between about 30° C. and about 500° C. and pressure range of between about 1 atmosphere and 10 atmospheres of pressure. It is generally preferred to incorporate a catalyst to improve rate of product diene production. The various catalysts that enable the alcohol-to-olefins conversion include, but are not limited to catalysts selected from: zeolites, supported transition metals, supported noble metals, supported rare earth metals, supported mixtures of transition, rare earths, and/or noble metals. Catalyst transition metals include Fe, Co, Cu, Zn, V, Ni, Ti, Cr, Mn, Re, Y, Zr, Mo, and Ta. Catalyst rare earth elements include La, Ce, Gd, Sc, Pr, Nd, Sm, Eu, Pr, Tb, Dy, Ho, Er, Tm, Yb and Lu. Catalyst noble metals include Pt, Pd, Rh, Ru, Au, Ir and Ag. Operable supports nonexclusively include: zeolites, alumina, silica and carbon. Other operable catalyst types include ion exchange resins. The catalysts can further be physical mixtures of more than one catalyst, catalyst and support, or support and support.

### EXAMPLE 1

### Methylbutenol Production

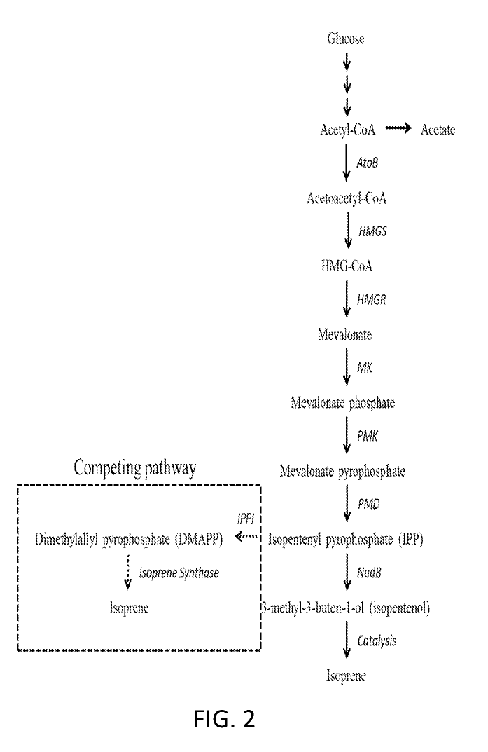

The production of 5-carbon alcohols (methylbutenol) from E. coli leverages the mevalonate pathway co-expressed with several synthetic enzymatic steps resulting in isopentenyl pyrophosphate (IPP) as an intermediate molecule. IPP is commonly used by cells as a precursor in the synthesis of quinones, cell membrane molecules, and higher order terpenes in some organisms. From these synthetic pathways, it is possible to favor production of 3-methyl-3-butenol or 3-methyl-2-butenol depending on the specificity and kinetics of the isomerase (IPPI) selected (see FIG. 2).

Since methylbutenols are not a natural product of E. coli, the pathway to their synthesis is desirably engineered and placed within a production E. coli organism. A 7-step pathway starting from acetyl-CoA and ending at methylbutenol was placed on two separate plasmids and transformed into an E. coli host. Extensive modification of the pathway was undertaken in order to yield a balanced pathway, which does not accumulate large fractions of any intermediate compounds that can have negative effects on the health of the cells and the upstream enzymes. The resultant production organism is termed strain 3A. The engineered pathway encompasses the mevalonate pathway transferred into E. coli with added hydrolase NudB to convert IPP to methylbutenol. Plasmid organization includes variants of the first 3 steps termed “top” and next two steps termed “bottom”. A second high copy number plasmid contains the last two enzymes NudB and PMD which remained unchanged.

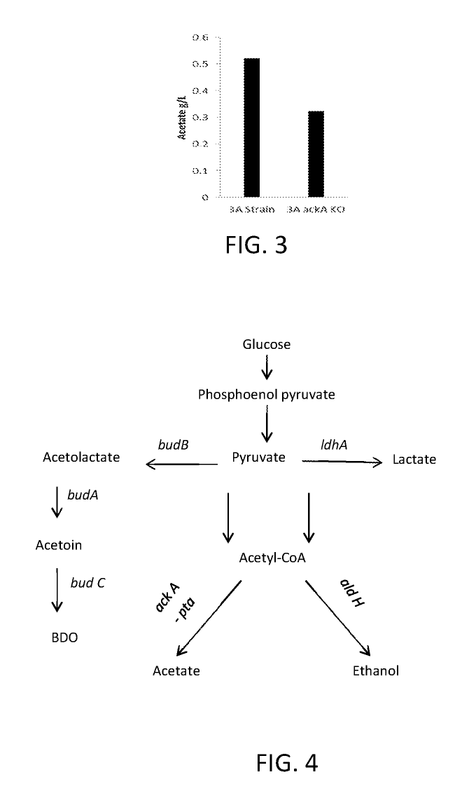

An initial strain of E. coli was demonstrated capable of producing 1.5 g/L from 10 g/L of glucose, which equates to a 46% yield compared to the theoretical maximum. The initial strain was obtained from Lawrence Berkeley National Laboratory (LBNL) for minimal media and biomass hydrolysate tolerance testing. Subsequently, an additional strain with changes in the promotor sequences and ribosome binding sites of several of the genes to further balance the engineered pathway resulted in a strain capable of producing 2.23 g/L from 10 g/L of glucose termed KG1R10. The increased expression level obtained equated to 70% of the theoretical pathway yield. These results were obtained using rich media and the model carbon source pure glucose in shake flask culturing. The challenge was to increase the titer while maintaining efficiency of conversion and transferring the producing organism into minimal media and an industrial carbon source such as woody biomass hydrolysate. Additional engineering that was performed on the two strains included knockouts of half of the acetate producing pathway (gene ackA) and complete knockout of the lactate producing pathway in separate organisms. A marked reduction in acetate accumulation can be seen in strain 3A in FIG. 3. Similar reduction was seen in the lactate knockout strains. In future work the strains will be double knocked out to show the effect of no acetate or lactate accumulation on methylbutenol titer. Future work will additionally investigate the knock out of the ethanol-producing pathway to direct more carbon flux to the desired alcohol products.

### 2,3-Butanediol (BDO) Production

Wild type Klebsiella oxytoca is known to produce high concentration of BDO, however with poor carbon yield. The metabolic diagram is shown in FIG. 4. Glucose is converted to pyruvate through several steps, and pyruvate is converted to acetolactate, acetoin, and BDO with acetolactate synthase (budB), acetolactate decarboxylase (budA), and acetoin reductase (budC), respectively. In addition, lactate, ethanol, and acetate are formed as side products leading to yield loss. Further, the native culture utilizes xylose in a lag phase.

Redirecting carbon flux: Metabolic engineering of K. oxytoca has been performed to eliminate competitive pathways for lactic acid and ethanol production thereby directing significantly higher flux to BDO. Elimination of ldhA and aldA genes has been performed to enhance BDO productivity. The Kan/FRT protocol was used to knock out genes originally developed by Datsenko and Wanner (Datsenko, Kirill A., and Barry L. Wanner. “One-step inactivation of chromosomal genes in Escherichia coli K-12 using PCR products.”Proceedings of the National Academy of Sciences 97.12 (2000): 6640-6645, hereby incorporated by this reference as a portion of this disclosure), however other knockout procedures can be used including CRISPR/Cas9, zinc finger nucleases, or other DNA or RNA based silencing or knockout procedures.

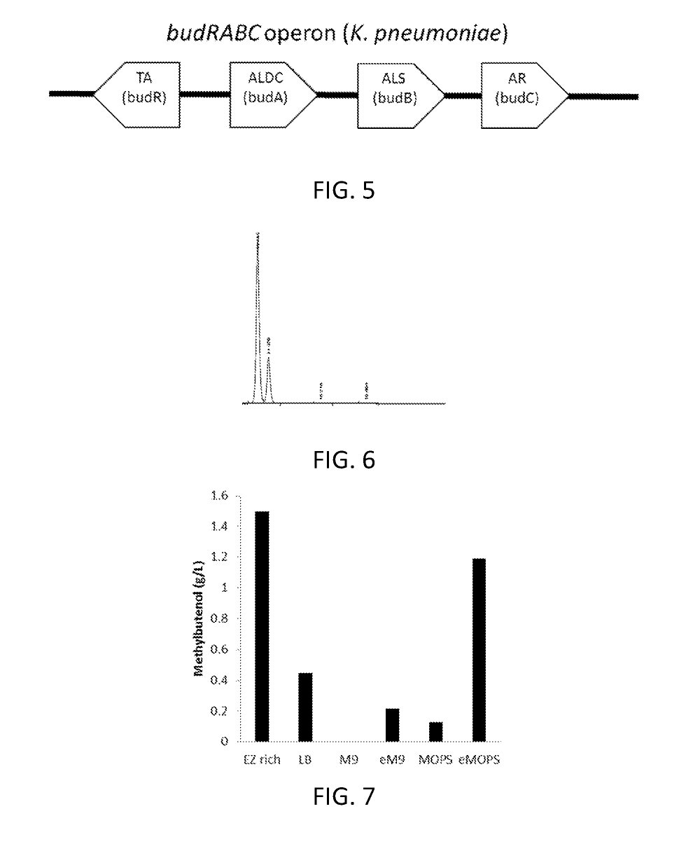

Overexpression of BDO pathway genes: An operon for BDO synthesis, budRABC, from K. pneumoniae is used to overexpress budA, budB, budC, and budR genes, to improve BDO productivity. These four genes are cloned as an operon into the pTrc99A vector (see FIG. 5).

Adaptation of K. oxytoca towards inhibitor tolerance: It has been demonstrated that certain K. oxytoca strains are tolerant to commonly occurring polyaromatic inhibitors in biomass hydrolysate.

Glucose is usually utilized as a feedstock for bacterial fermentation. Use of woody biomass hydrolysate, with a mixture of C6 and C5 sugars, has been demonstrated. Industrially produced biomass hydrolysate was obtained from a third party. Initial feasibility was demonstrated by using glucose as a model sugar. However, for commercial feasibility, the bioprocessing should utilize hydrolysate to be cost competitive with chemical routes of synthesis. FIG. 6 shows an exemplary high performance liquid chromatography (HPLC) plot of woody biomass derived hydrolysate.

Though the original development of the strains for the production of 5-carbon alcohols was performed in rich media with the ideal carbon source pure glucose, the conversion to a minimal media formulation and industrial carbon sources was vital for the ability to scale up the technology. Initially the carbon source and concentration was varied using the rich media as a base. Glucose was tested against fructose and glycerol as the different carbon sources are known to often affect the metabolites produced from each organism. Glucose was shown to be the most effective carbon source with fructose and glycerol generating significantly less titer of methylbutenol per gram supplemented. Multiple minimal medias were also attempted and compared to LB media and EZ rich media, which are the rich media standards, including M9 minimal media (M9), enhanced M9 minimal media (eM9), 3-morpholinopropanesulfonic acid (MOPS) minimal media (modified Neidhardt), and enhanced MOPS minimal media (eMOPS). The goal was to shift to a minimal media for culturing with minimal loss in methylbutenol titer achieved using rich media. Each of the minimal mediums along with EZ rich were supplemented with 10 g/L of glucose as a carbon source. FIG. 7 shows the results of these trials. M9 was unable to support bacterial growth without supplementation, and as a result was unable to produce any methylbutenol titer. The eM9 and MOPS medias performed similarly with under 0.25 g/L methylbutenol produced while the eMOPS produced nearly 80% of the titer of EZ rich at 1.19 g/L. As a result eMOPS was chosen as the media moving forward into fermentation bioprocess development. At the shake flask level of carbon source supplementation, and double the shake flask concentration of 20 g/L glucose there was no inhibition of cell growth due to any impurities present in the industrially produced woody biomass hydrolysate.

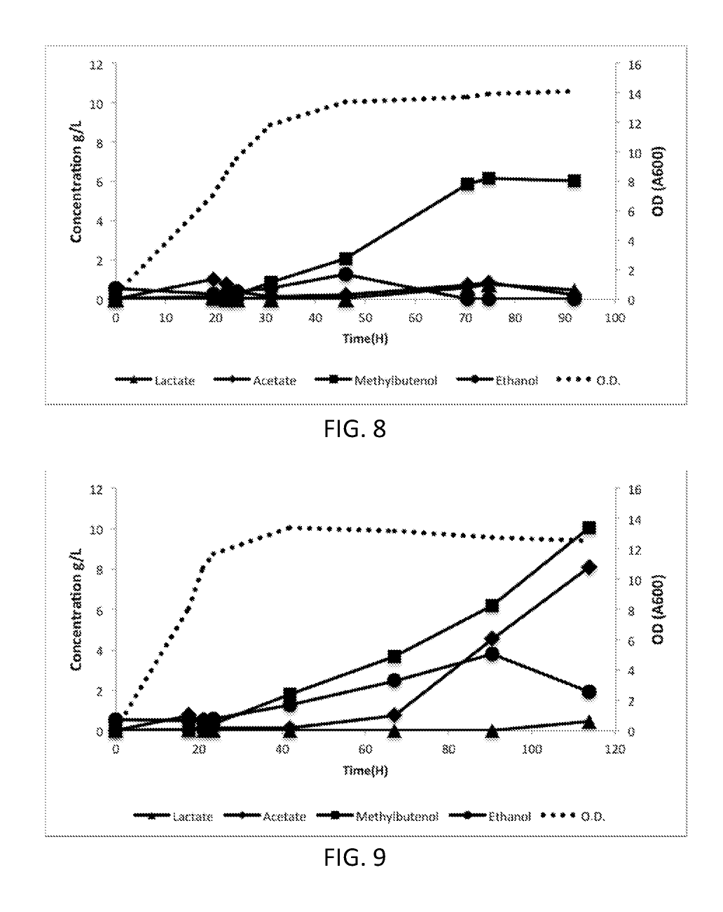

Following strain development, media formulation, and industrial feedstock tolerance testing, the process was scaled up to a 10 L reactor to determine initial operating parameters and ensure methylbutenol production could be maintained in a fermentation environment. The initial operating parameters were determined through previous experience in a variety of expression systems including manufacture of BDO from K. oxytoca and free fatty acids from E. coli. Temperature of the culture was set at 30° C. as lowering the culture temperature from 37° C. often aids in plasmid stability and increased expression. Agitation and airflow rates were chosen to simulate the best-case scenario in shake flask testing. The fermenter controller was set to maintain a pH of 7.0 through addition of 25% ammonium hydroxide as is often optimal for E. coli expression systems. Any excess foam was controlled via the addition of 1% antifoam 204. The nutrient and carbon source starting conditions were cloned from the highest expressing shake flask culture systems. Feeding was achieved through constant addition of biomass hydrolysate at a rate of approximately 1 g/L/hr, but was adjusted as necessary to maintain a glucose concentration between 1 g/L and 10 g/L. Glucose concentration was periodically measured via glucometer during the fermentation and HPLC after the completion of the fermentation. Complicating this task is the toxic effect of the phenolic and polyaromatic hydrocarbons that are typically present in biomass hydrolysate. The buildup of these compounds alters the metabolism of the production organism leading to changing glucose consumption rates. With these conditions a methylbutenol concentration of 6.13 g/L was achieved with limited buildup of common byproducts: acetate, lactate, and ethanol though it is postulated that some of the acetate and ethanol were removed from the system via evaporation and entrainment (FIG. 8). The efficiency achieved was 58% of theoretical maximum. With additional optimization, a longer carbon source feed, and secondary capture mechanisms in place to minimize loss due to entrainment and evaporation the achieved titer increased to 10.02 g/L of methylbutenol at an efficiency of 65% of the theoretical maximum (FIG. 9). Changes in the airflow pathway as well as increased variability in glucose concentration lead to an increased buildup of acetate, though this can be controlled through the use of knockout strains.

### Fermentation to Produce Butanediol

Wild type K. oxytoca was cultured with 7% glucose in fed-batch culturing at shake-flask level to demonstrate organism robustness. BDO formation of 9% was demonstrated after 4 batches. BDO quantity was observed to continue to increase even after 4 cycles. Thus, it can be deduced that BDO culturing has no feedback inhibition at moderate concentrations. With this initial discovery, the team has taken the approach of metabolic engineering to maximize BDO yield and titer. The theoretical carbon yield is 0.5. A carbon yield of 70% of theoretical has been demonstrated thus far.

Fermentation with Lactate Elimination Strains

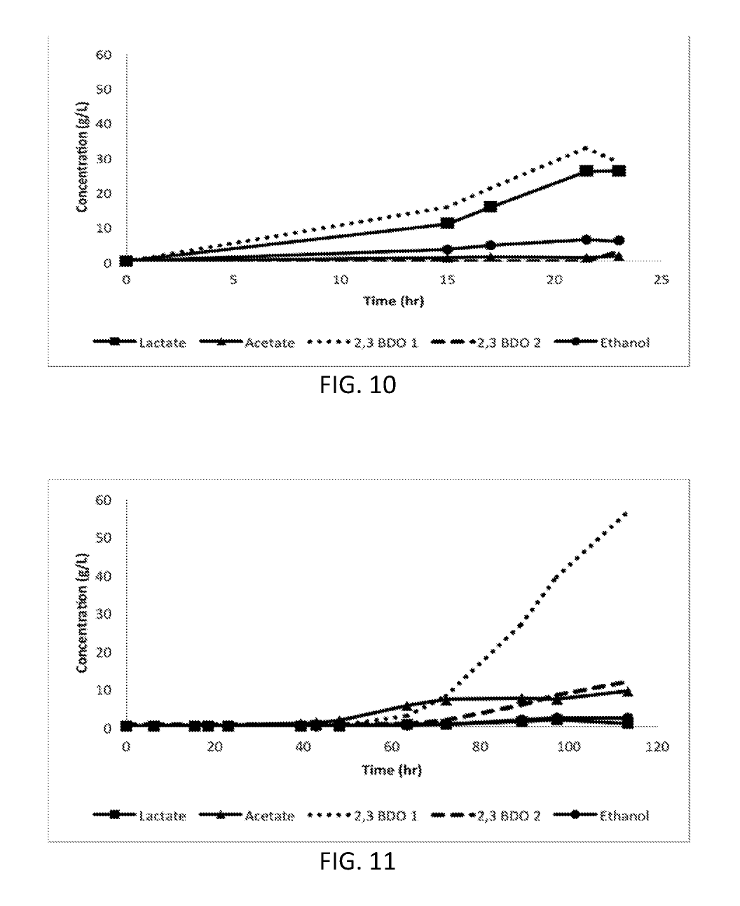

The experiment was performed in a 3 L fermenter. Aeration flow rate of 1 LPM/L of culture was used initially with a drop to 0.4 LPM/L of culture after initial growth conditions to trigger a micro-aerobic state and increase expression of BDO. The hydrolysate selection events were triggered through gradual use of high concentrations of wood hydrolysate whereas typical reports only recognize use of up to 5% hydrolysate due to growth inhibition by vanillin and aromatic compounds (see FIGS. 10 and 11).

Separation from Water

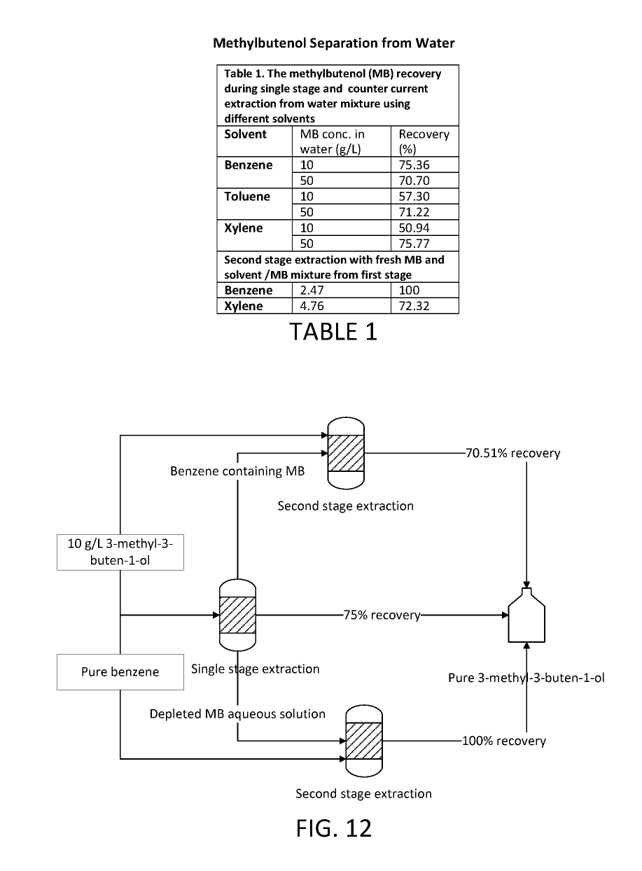

In most of the fermentation-based processes, since the product formed is in excess water, separation has been identified as an expensive unit operation. In contrast to ethanol, butanol, fatty acids, or lactic acid type of fermentations, the proposed innovation in one particular embodiment produces an unsaturated product methylbutenol (one double bond). This feature is exploitable to enable a very low cost extraction based separation process. Once the methylbutenol has been extracted from aqueous phase via organic solvents, it can be directly converted to isoprene, a very low boiling compound (35° C.), and can be collected as overhead from the dehydration reactor. Alternatively, the separation of methylbutenol and organic solvents is trivial distillation. A number of experiments were performed with different solvents to establish extraction based separation feasibility. Organic solvents, namely, benzene, toluene, and xylene were tested for extraction efficiency. The methylbutenol concentrations in water were chosen to be 10 g/L (established) and 50 g/L (future possibility). Single pass partitioning of up to 75% has been established by use of benzene at room temperature. Second pass extraction with fresh solvent essentially completes the extraction with nearly 100% partitioning of methylbutenol in benzene. Experimental details are shown in Table 1. FIG. 12 shows the two-pass extraction scheme demonstrating 100% recovery of methylbutenol from excess water with benzene as the solvent.

### Catalytic Conversion of Methylbutenol to Isoprene

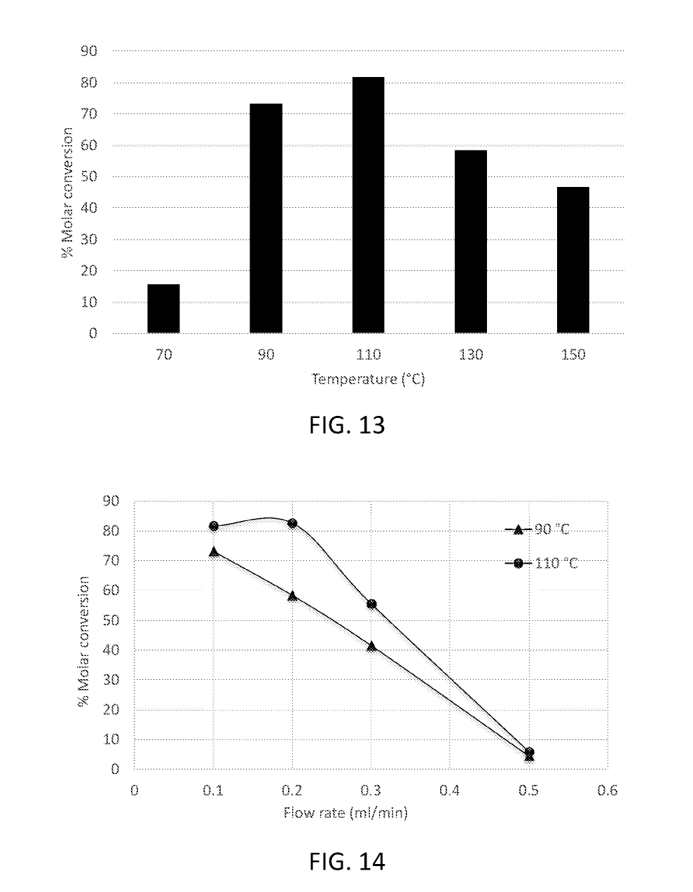

A number of experiments were performed with industrial Amberlyst® catalysts. The temperature was varied from 70° C. to 150° C., Liquid hourly space velocity (LHSV) was varied between 6 and 18 per hour. A total of 1 gm of catalyst was loaded in a tubular reactor heated by furnace (ATS) with proportional integral derivative (PID) controller. Expected operable catalytic temperature and pressure range is 70-220 C and 1-10 atm respectively. FIGS. 13 and 14 show the results of single pass conversion at LHSV of 12 per hour. It can be observed that, conversion is maximum at 110° C., which is below the boiling point of methylbutenol. Thus, it was concluded that the dehydration reaction is liquid phase. The formed isoprene was simply decanted from unreacted methylbutenol and formed water. The total collected isoprene was measured to estimate total conversion.

### Catalytic Conversion of Butanediol to Butadiene

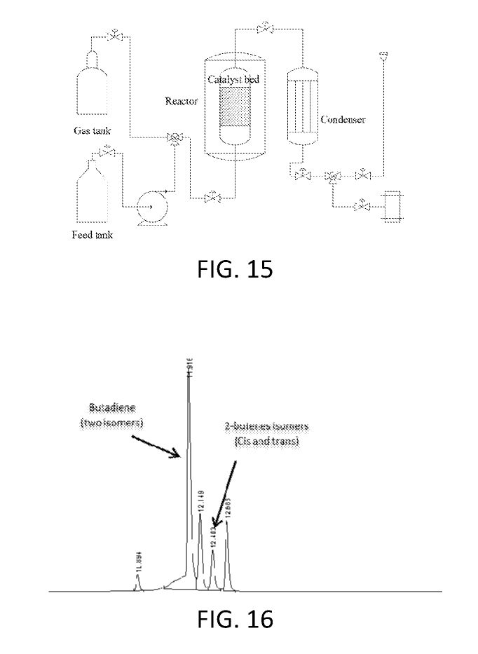

Experiments have been conducted on conversion of BDO using different catalysts. The conversion reaction was carried out in a flow apparatus similar to that shown in FIG. 15. The solution of BDO was introduced in the stainless steel reactor by positive displacement pump at flow rates of 1.08 ml/h and reaction was carried out at a temperature of 450° C. The formed products were sent to a condenser and collected periodically by opening valve in sample collection bottle. The liquid samples were pooled together and distilled at temperature of 70-75° C., 80-120° C., and 135° C. and above. Different distillation fractions were analyzed for methylethylketone and other hydrocarbons with unreacted BDO using gas chromatography. The gas samples were collected from exhaust and were directly analyzed for 1,3-butadiene and 2-butene concentration using gas valve fitted gas chromatography. FIG. 16 shows the GC-FID chromatogram with identified 1,3-butadiene and 2-butene fractions.

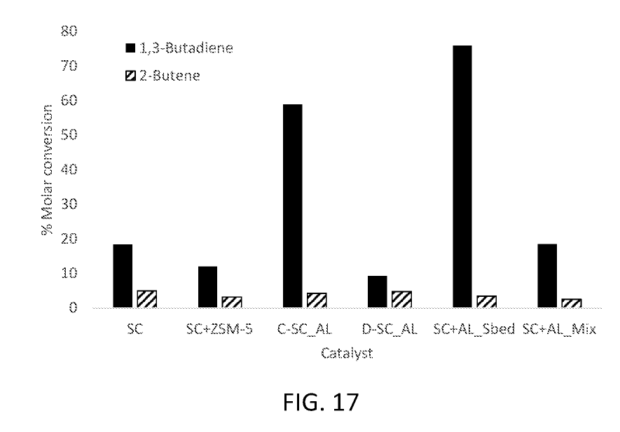

FIG. 17 shows the BDO dehydration results of different catalysts at 1.08 ml/hr flow rate and reaction temperature of 450° C. During dehydration of BDO it was observed that single catalyst scandium oxide does not work efficiently and maximum molar conversion observed was about 18%. These results are in contradiction to the literature report of Duan et al. (Duan H, Yamada Y, Sato S, Efficient production of 1,3-butadiene in the catalytic dehydration of 2,3-butanediol, Applied Catalysis A: General 491 (2015) 163-169) who observed Sc₂O₃ to produce 88.3% of 1,3-butadiene at around 411° C. A composite catalyst was prepared by depositing scandium on alumina surface. Two catalysts identified as C_SC_AL (concentrated scandium on alumina contains about 0.19 gm of scandium/gm of alumina) and D_SC_AL (dilute scandium on alumina contains about 0.10 gm of scandium/gm of alumina) were prepared using the incipient wetness method. The concentrated scandium immobilized on alumina obtained about 59% conversion of BDO to 1,3-butadiene. One of the experiments was performed by preparing separate beds of 1 gm scandium and alumina identified as SC+AL. One bed showed net single pass conversion of 75%. This demonstrates the feasibility of catalytic conversion of BDO to 1,3-butadiene using a bifunctional catalyst from rare earth group and acidic function.

Although the invention has been described with regard to certain preferred embodiments, the scope of the invention is to be encompassed by the appended claims.

What is claimed is:

1. A method to produce a diene molecule, comprising the steps of:
providing a carbon-bearing biomass;
converting the biomass to biomass hydrolysate;
fermenting the biomass hydrolysate to produce fermentation broth comprising of an alcohol with at least two different reactive sites or two hydroxyl groups;
separating the alcohol from fermentation broth; and
catalyzing the alcohol to form the diene molecule.

2. The method according to claim 1, wherein:
the step of converting the biomass to a biomass hydrolysate comprises pretreating the biomass to initiate breakdown of the biomass material.

3. The method according to claim 1, wherein:
the step of converting the biomass to a biomass hydrolysate comprises hydrolyzing the biomass to produce monomeric sugars carried in a liquid solvent.

4. The method according to claim 1, further comprising the step of:
obtaining an engineered organism that can feed on the biomass hydrolysate and subsequently express a desired alcohol product.

5. The method according to claim 4, wherein:
the step of fermenting the biomass hydrolysate comprises adding the engineered organism to the fermentation mixture.

6. The method according to claim 5, wherein:
the organism is selected from the group comprising (prokaryotic and eukaryotic organisms).

7. The method according to claim 5, wherein:
the organism is selected from the group comprising (bacteria, yeast, fungi, archaea, cyanobacteria, insect, plant, and mammalian cells).

8. The method according to claim 5, wherein:
the organism is selected from the group comprising (gram-positive bacterial cells, gram-negative bacterial cells, filamentous fungal cells, algae cells, and yeast cells).

9. The method according to claim 5, wherein:
the organism is selected from the group comprising (Escherichia sp. (E. coli), Panteoa sp. (P. citrea), Bacillus sp. (B. subtilis), Yarrowia sp. (Y. lipolytica), Saccharomyces sp. (S. cerevisiae), Pichia sp. (P. pastoris), Trichoderma sp. (T. reesei), Aspergillus sp. (A. oryzae or A. niger), Klebsiella sp. (K. oxytoca or K. pneumoniae), Streptomyces sp. (S. lividans or S. californicus), Clostridium sp. (C. ljungdahlii), Enterobacter sp. (E. aerogenes), Aerobacillus sp. (A. polymyxa), Lactococcus sp. (L. lactis), Paenibacillus sp. (P. polymyxa), Serrati sp. (S. marcescens), Candida sp. (C. rugosa), Geobacillus sp. (G. thermoglucosidasius), Serratia sp. (S. plymuthica), Pyrococcus sp. (P. furiosus), Corynebacterium sp. (C. glutamicum), and Pseudomonas sp. (P. aeruginosa)).

10. The method according to claim 1, wherein the step of separating the alcohol from the fermentation broth comprises one or more process selected from the group comprising (distillation, filtration, solvent extraction, membrane separation, pervaporation, absorption, adsorption, vacuum distillation, and use of adducts).

11. The method according to claim 1, wherein the step of separating the alcohol from the fermentation broth comprises solvent extraction, and the solvent is selected from the group comprising (methyl iso-butyl ketone, methyl ethyl ketone, acetone, ethanol, propanol, hexane, butyl acetate, ethyl acetate, benzene, toluene, xylene, N-Methyl-2-pyrrolidone, glycerol, glycol, cyclohexane, chloroform, dichloromethane, ethyl acetate, dimethyl formamide, acetonitrile, dimethyl sulphoxide, and butanol).

12. The method according to claim 1, wherein the step of separating the alcohol from the fermentation broth comprises:
centrifugal separation of solids from a liquid portion of fermented broth;
solvent extraction of the alcohol from the fermented broth; and
temperature-based selective evaporation and product collection by condensation.

13. The method according to claim 1, wherein the step of catalytically converting the alcohol to form the diene molecule is performed in a continuous stirred tank or in a packed bed reactor.

14. The method according to claim 1, wherein the step of catalytically converting the alcohol to form the diene molecule is performed in the temperature range of between about 30° C. and about 500° C. and in the pressure range of between about 1 atmosphere and about 10 atmospheres of pressure.

15. The method according to claim 1, wherein the step of catalytically converting the alcohol to form the diene molecule is carried out in the presence of a catalyst.

16. The method according to claim 15, wherein the catalyst includes one or more element selected from the group comprising (zeolites, supported transition metals, supported noble metals, supported rare earth metals, supported mixtures of transition, rare earths, noble metals, and ion exchange resin).

17. The method according to claim 1, wherein:
the diene molecule is isoprene.

18. The method according to claim 1, wherein:
the diene molecule is butadiene.

19. A method to produce an isoprene molecule, comprising the steps of:
providing a carbon-bearing biomass;
converting the biomass to a biomass hydrolysate;
obtaining an engineered organism that can feed on the biomass hydrolysate and subsequently express a desired alcohol product comprising methylbutenol;
using the engineered organism to ferment the biomass hydrolysate and produce fermentation broth comprising of the desired alcohol product;
separating the alcohol product from the fermentation broth; and
catalytically converting the alcohol product to form the isoprene molecule.

20. A method to produce a butadiene molecule, comprising the steps of:
providing a carbon-bearing biomass;
converting the biomass to a biomass hydrolysate;
obtaining an engineered organism that can feed on the biomass hydrolysate and subsequently express a desired alcohol product comprising 2,3-butanediol;
using the engineered organism to ferment the biomass hydrolysate to and produce fermentation broth comprising of the desired alcohol product;
separating the alcohol product from fermentation broth; and
catalytically converting the alcohol product to form the butadiene molecule.
